(19) United States
(12) Patent Application Publication
(10) Pub. No.: US 2023/0310528 A1
(43) Pub. Date: Oct. 5, 2023

(54) DEEP EUTECTIC SOLVENT INCLUDING ONE OR MORE ACTIVE PHARMACEUTICAL INGREDIENTS DERIVED FROM MUSHROOMS

(71) Applicant: Optimi Health Corp., Vancouver (CA)

(72) Inventor: Justin Kirkland, Champaign, IL (US)

(21) Appl. No.: 18/095,298

(22) Filed: Jan. 10, 2023

Related U.S. Application Data
(60) Provisional application No. 63/299,750, filed on Jan. 14, 2022.

(51) Int. Cl. A61K 36/06; A61K 45/06; A61K 31/6615; A61K 9/70

(52) U.S. Cl. CPC A61K 36/06; A61K 9/7038; A61K 31/6615; A61K 45/06

(57) ABSTRACT

Disclosed herein is a two-part processing method that involves the extraction and transdermal delivery of psilocybin active components.

### CROSS REFERENCE TO RELATED APPLICATIONS

The present application claims the benefit of the filing date of U.S. Provisional Pat. Application No. 63/299,750 filed on Jan. 14, 2022, the disclosure of which is hereby incorporated by reference herein in its entirety.

### BACKGROUND OF THE DISCLOSURE

Mushrooms containing psilocybin (so-called magic mushrooms) are a group of psychoactive mushrooms, also known as magic mushrooms or hallucinogenic mushrooms. Mushrooms belonging to this group, such as Psilocybe cyanescens, also known as blue bald head, contain the psychedelic (mind-expanding, hallucinogenic) substances psilocybin and psilocin, which are being researched as pharmaceutically active ingredients. Psilocybin itself is not psychoactive - rather it is the dephosphorylated derivative psilocin that causes the hallucinogenic effect. Psilocybin is rapidly dephosphorylated to psilocin following ingestion in the mucosa by alkaline phosphatases and nonspecific esterases. Psilocin is structurally similar to human signaling molecules such as serotonin, and has been shown to bind to over 15 human serotonin-related receptors.

Scientific interest in classic psychedelics, such as psilocybin, has returned and grown because of several promising studies, validating earlier research. For mood and anxiety 1:1

disorders, three controlled trials have suggested that psilocybin may decrease symptoms of depression and anxiety in the context of cancer-related psychiatric distress for at least 6 months following a single acute administration. A small, open-label study in patients with treatment-resistant depression showed reductions in depression and anxiety symptoms three months after two acute doses. For addiction, small, open-label pilot studies have shown promising success rates for both tobacco and alcohol addiction. Safety data from these various trials, which involve careful screening, preparation, monitoring, and follow-up, indicate the absence of severe drug-related adverse reactions (Source: Johnson & Griffiths, 2017). Based on a recent report by Data Bridge Market Research, Market Analysis Study, 2020, the psychedelic drug market is growing with a compound annual growth rate of 16.3% and is expected to reach USD 6,859.95 million by 2027.

Clinical trials have recently recognized psilocybin as a promising candidate for the treatment of various psychological and neurological afflictions. Preliminary results suggest that psilocybin assisted treatment may be a good candidate for managing substance addiction (Bogenschutz et al., 2015; Riaz et al., 2016), anxiety in terminally ill patients (Grab et al., 2011), cluster headaches (Tyls et al., 2014), and treatment- resistant depression (Carhart-Harris et al., 2018). Psilocybin seems to be a particularly interesting candidate for “treatment resistant depression” - a term applied to the 13% of patients with Major Depressive Disorder (MDD) who relapse, in spite of four rounds of traditional treatment (Rush et al., 2006). Approximately 16 million Americans carried the MDD diagnosis in 2016, indicating a large number of people with untreated mental illness (Tice, 2017). Unfortunately, the content of psilocybin and psilocin in hallucinogenic mushrooms is too low (0.2%-1% dry weight) to make extraction a commercially viable option (Tyls et al., 2014), and chemical synthesis is complicated and expensive (Nichols and Frescas, 1999). Although the chemical synthesis of psilocybin has been improved since its discovery by Hoffman et al. in 1959, who achieved final yields of 20% of semi pure psilocybin, it continues to challenge chemists primarily due to the difficulty of the last synthetic step; the phosphorylation of psilocin (Nichols and Frescas, 1999).

Topical and transdermal drug delivery provide many advantages over other common delivery routes like oral, subcutaneous, and intravenous. These advantages include avoidance of major degradative pathways associated with the GI tract, reduction in side effects associated with systemic toxicity, and needle-free drug administration. Brown, et al., “Dermal and transdermal drug delivery systems: current and future prospects,” Drug Delivery, 13:175-87 (2006).

Transdermal absorption is a recognized means of systemic drug administration which benefits from being a non-invasive and convenient way of medicating a patient. Transdermal absorption is a potentially useful means of drug administration for patients who find other methods difficult or unpleasant. For example, the young and the old can have difficulty with orally administered medications and may find injections particularly unpleasant. Children and patients with dementia can also be difficult to medicate due to lack of compliance. As such, transdermal administration of therapeutic agents could be a valuable method of administering a medication, especially in the young, the old or mentally impaired patients.

### BRIEF SUMMARY OF THE DISCLOSURE

In one aspect of the present disclosure is as an improved process for the extraction of one or more active pharmaceutical ingredients (APIs) found in a fungal biomass. In some embodiments, the one or more APIs found in the fungal biomass may be extracted using a deep eutectic solvent or a deep eutectic solvent system. In some embodiments, the extraction provides for a pharmaceutical composition comprising the one or more APIs and the deep eutectic solvent or the deep eutectic solvent system. In some embodiments, the pharmaceutical compositions do not include any further additives. In some embodiments, the pharmaceutical composition may be administered directly to a subject in need of treatment thereof. In some embodiments, the pharmaceutical composition may be administered directly to a subject in need of treatment thereof, such as without the use of a penetration or permeation enhancer. In some embodiments, the pharmaceutical composition is administered transdermally or to a mucous membrane.

Another aspect of the present disclosure is a pharmaceutically acceptable formulation comprising the pharmaceutical composition described above and one or more pharmaceutically acceptable carriers or excipients, i.e., a pharmaceutically acceptable formulation comprising (i) the one or more APIs, (ii) the deep eutectic solvent or the deep eutectic solvent system; and (iii) one or more pharmaceutically acceptable carriers or excipients. In some embodiments, the pharmaceutically acceptable formulation is an external preparation for transdermal administration.

Another aspect of the present disclosure is a process for the extraction of heterocyclic compounds found in mushrooms, e.g., compounds naturally produced by the mycelium of psilocybin mushrooms. Examples of such compounds include baeocystin, norbaeocystin, N,N-dimethyltryptamine, 5-hydroxytryptamine (serotonin), 5-hydroxytryptophan, psilocybin and psilocin. In some embodiments, the compounds found in the mushrooms may be extracted using a deep eutectic solvent or a deep eutectic solvent system. In some embodiments, a pharmaceutical composition comprising the deep eutectic solvent or the deep eutectic solvent system and the extracted heterocyclic chemicals is applied directly to the skin of a patient in need of treatment (such as without the inclusion of any other additional components). In some embodiments, the pharmaceutical compositions described herein are topically applied to the skin with negligible or no skin irritation (as evidenced by redness, burning and/or itching sensations). In some embodiments, the pharmaceutical compositions contain neat ionic liquids in combination with the extracted heterocyclic compounds. In some embodiments, the compositions are applied topically to the surface of the skin without the use of penetration enhancers or permeation enhancers.

In some embodiments, the pharmaceutical composition comprising the deep eutectic solvent or the deep eutectic solvent system and the extracted heterocyclic chemicals is combined with one or more pharmaceutically acceptable carrier or excipients (to provide a pharmaceutically acceptable formulation, such as one suitable for transdermal administration to a subject in need of treatment thereof). Suitable pharmaceutically acceptable carrier or excipients include, but are not limited to, penetration or permeation enhancers, plasticizers, gelling agents, fluid carriers, tackifiers, cohesion-promoting additives, stabilizers, fillers, colorants, dyes, UV-absorbing compounds, antioxidants and similar additives.

Another aspect of the present disclosure is a pharmaceutical composition comprising a deep eutectic solvent and one or more APIs derived from a fungal biomass. In some embodiments, the fungal biomass comprises psilocybin mushrooms. In some embodiments, the one or more APIs include at least one serotonin agonist. In some embodiments, the one or more APIs are selected from the group consisting of include baeocystin, norbaeocystin, N,N-dimethyltryptamine, 5-hydroxytryptamine (serotonin), 5-hydroxytryptophan, psilocybin and psilocin.

In some embodiments, the deep eutectic solvent comprises a first liquid component and a second liquid component. In some embodiments, the first liquid component is choline chloride. the second liquid component is selected from the group consisting of a sugar, an allylene glycol, a diol, and an acid. In some embodiments, a ratio of the first liquid component to the second liquid component ranges from 5:1 to 1:5. In some embodiments, the ratio ranges from 4:1 to 1:4. In some embodiments, the ratio ranges from 3:1 to 1:3. In some embodiments, the ratio ranges from 2:1 to 1:2. In some embodiments, the ratio is about 1 to about 1.

In some embodiments, the deep eutectic solvent further comprises a third liquid component. In some embodiments, the third liquid component is selected from the group consisting of xylitol or proline.

In some embodiments, the deep eutectic solvent is selected from the group consisting of a mixture of Choline Chloride and Glucose, a mixture of Choline Chloride and Fructose; a mixture of Choline chloride and ethylene glycol; a mixture of Choline chloride and 1,2-propanediol; a mixture of Choline Chloride and Glycerol; a mixture of Choline chloride and 1,4-butanediol; a mixture of Choline chloride and citric acid; a mixture of Choline Chloride and Malic Acid; a mixture of Choline chloride and Geranic acid; a mixture of Choline Chloride and Malonic Acid; a mixture of Choline Chloride and Urea; a mixture of Betaine and Glycerol; a mixture of Betaine and Citric acid; a mixture of Betaine and Malic acid; a mixture of Proline and Glycerol; a mixture of Lactic acid and glucose; and a mixture of and Choline chloride and Geranic Acid.

In some embodiments, the deep eutectic solvent is selected from the group consisting of a mixture of Choline Chloride, Malic Acid, and Xylitol; a mixture of Choline Chloride, Malic Acid, and Proline; and a mixture of Choline Chloride, Urea, and 1,2-Propanediol.

In some embodiments, the deep eutectic solvent is selected from the group consisting of a mixture of Betaine, Malic Acid, and Glucose; a mixture of Betaine, Proline, and Glucose; a mixture of Betaine, Proline, and Malic acid; and a mixture of Betaine, Proline, and Sucrose.

In some embodiments, the deep eutectic solvent comprises a mixture of dichloroacetic acid, 1-menthol, and n-butanol. In some embodiments, the deep eutectic solvent comprises a mixture of proline, malic acid, lactic acid, and water.

In some embodiments, the pharmaceutical composition further comprises one or more pharmaceutically acceptable carriers or excipients. In some embodiments, the one or more pharmaceutically acceptable carriers or excipients comprise penetration enhancers or permeation enhancers. In some embodiments, the penetration enhancers or permeation enhancers are selected from the group consisting of methyl laurate, ethyl oleate, glycerol mono oleate, oleic acid, oleyl alcohol, isopropyl palmitate, isopropyl myristate, octyldodecanol, ω-pendadecalactone, cyclopendadecanone, propylene glycol monolaurate, eucalyptol, Ceraphyl 31, 1-dodecanol, transcutol P, triacetin, propylene glycol, dipropylene glycol, butylene glycol, ethanol, octanol, limonene, sorbitan monooleate, n-alkylphenol ether ethoxylates, n-alkyl ether ethoxylates, and mixtures thereof.

Another aspect of the present disclosure is a method of treating a human subject comprising a therapeutically effective amount of a pharmaceutical composition comprising a deep eutectic solvent and one or more APIs derived from a fungal biomass to the human subject in need of treatment thereof. In some embodiments the administration is transdermal.

Another aspect of the present disclosure is a transdermal delivery system comprising (i) an adhesive layer including a pharmaceutical composition comprising a deep eutectic solvent and one or more APIs derived from a fungal biomass blended within an adhesive material, and (ii) a backing layer. In some embodiments, the transdermal delivery system further comprises a release liner.

Another aspect of the present disclosure is a monolithic suspension-blend transdermal patch comprising: (a) a backing layer having inner and outer surfaces and being substantially impervious to a pharmaceutical composition comprising a deep eutectic solvent and one or more APIs derived from a fungal biomass; (b) a first adhesive layer having a first surface covering at least a portion of the inner surface of the backing layer, the adhesive layer comprising a uniform suspension of a therapeutically-effective amount of the pharmaceutical composition comprising the deep eutectic solvent and the one or more APIs derived from a fungal biomass in an adhesive; (c) a removable release liner in contact with a second surface of the adhesive layer.

In some embodiments, the adhesive comprises a pressure sensitive adhesive. In some embodiments, the adhesive layer comprises between about 5% to about 35% of the pharmaceutical composition. In some embodiments, the adhesive is polyisobutylene.

Another aspect of the present disclosure is a cream or ointment comprising (i) a pharmaceutical composition comprising a deep eutectic solvent and one or more APIs derived from a fungal biomass; and (ii) a fatty oil, a lanolin, a petrolatum, a paraffin, a plastibase, one or more glycols, a higher fatty acid, a higher alcohol, or any combination thereof.

Another aspect of the present disclosure is a lotion comprising (i) a pharmaceutical composition comprising a deep eutectic solvent and one or more APIs derived from a fungal biomass; and (ii) ethanol, glycerine, glycol, or any combination thereof.

Another aspect of the present disclosure is a gel comprising (i) a pharmaceutical composition comprising a deep eutectic solvent and one or more APIs derived from a fungal biomass; and (ii) a product obtained by gelatinizing liquid fats and oils by a gelling agent for an oily gel preparation, or a gelling agent such as a carboxymethyl polymer, a hydroxypropylcellulose or a polyvinyl alcohol for an aqueous gel preparation.

Another aspect of the present disclosure is a method of treating a patient suffering from anxiety, depression, and/or other mental health conditions comprising contacting the skin of the patient in need of treatment thereof with any of the above-identified transdermal delivery systems, monolithic suspension-blend transdermal patches, creams, ointments, lotions, or gels.

Another aspect of the present disclosure is a mixture comprising (i) a deep eutectic solvent, and (ii) a fungal biomass which has been cut, sheared, minced, or sliced. In some embodiments, the deep eutectic solvent is selected from the group consisting of a mixture of Choline Chloride and Glucose, a mixture of Choline Chloride and Fructose; a mixture of Choline chloride and ethylene glycol; a mixture of Choline chloride and 1,2-propanediol; a mixture of Choline Chloride and Glycerol; a mixture of Choline chloride and 1,4-butanediol; a mixture of Choline chloride and citric acid; a mixture of Choline Chloride and Malic Acid; a mixture of Choline chloride and Geranic acid; a mixture of Choline Chloride and Malonic Acid; a mixture of Choline Chloride and Urea; a mixture of Betaine and Glycerol; a mixture of Betaine and Citric acid; a mixture of Betaine and Malic acid; a mixture of Proline and Glycerol; a mixture of Lactic acid and glucose; and a mixture of and Choline chloride and Geranic Acid.

Another aspect of the present disclosure is a liquid extracted from or filtered from the mixture described above, wherein the liquid includes the deep eutectic solvent and one or more APIs derived from the fungal biomass. In some embodiments, the one or more APIs are selected from the group consisting of include baeocystin, norbaeocystin, N,N-dimethyltryptamine, 5-hydroxytryptamine (serotonin), 5-hydroxytryptophan, psilocybin and psilocin.

Another aspect of the present disclosure is a method of treating a patient suffering from anxiety, depression, and/or other mental health conditions comprising contacting the skin of the patient in need of treatment thereof with the liquid describes above.

Another aspect of the present disclosure is an adhesive blend comprising the liquid described above and an adhesive.

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

### Definitions

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of.” “Consisting essentially of,” when used in the claims, shall have its ordinary meaning as used in the field of patent law.

As used herein, the terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “administering” refers to the physical introduction of a composition comprising a therapeutic agent to a subject, using any of the various methods and delivery systems known to those skilled in the art. Routes of administration for the formulations disclosed herein include transdermal. intravenous, intramuscular, subcutaneous, intraperitoneal, spinal or other parenteral routes of administration, for example by injection or infusion.

As used herein, the terms “patient” and “subject”, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Paolo ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human in need of treatment of cancer, including through inhibiting the proliferation or viability of cancer cells and/or elicitation of an immune response (e.g., an immune response with enhanced T-cell activation) to cancer cells. However, it will be understood that the aforementioned terms do not imply that symptoms are present.

As used herein, the term “psilocybin” refers to a naturally occurring psychedelic prodrug compound produced by more than 200 species of mushrooms, collectively known as psilocybin mushrooms. The most potent are members of the genus Psilocybe, such as P. azurescens, P. semilanceata, and P. cyanescens, but psilocybin has also been isolated from about a dozen other genera. As a prodrug, psilocybin is quickly converted by the body to psilocin, which has mind-altering effects similar, in some aspects, to those of LSD, mescaline, and DMT. In general, the effects include euphoria, visual and mental hallucinations, changes in perception, a distorted sense of time, and spiritual experiences, and can also include possible adverse reactions such as nausea and panic attacks. The concentration of active psilocybin mushroom compounds (APIs) varies not only from species to species, but also from mushroom to mushroom inside a given species, subspecies or variety. The same holds true even for different parts of the same mushroom. In the species Psilocybe samuiensis, the dried cap of the mushroom contains the most psilocybin at about 0.23 wt. %-0.90 wt. %. The mycelium contains about 0.24 wt. %-0.32 wt. %.

As used herein, the term “psilocin” (also known as 4-HO-DMT, 4-hydroxy DMT, psilocine, psilocyn, or psilotsin) refers to a substituted tryptamine alkaloid and a serotonergic psychedelic substance. It is present in most psychedelic mushrooms together with its phosphorylated counterpart psilocybin.

As used herein, the term “baeocystin” refers to a psilocybin mushroom alkaloid and analog of psilocybin. It is found as a minor compound in most psilocybin mushrooms together with psilocybin, norbaeocystin, and psilocin. Baeocystin is an N-demethylated derivative of psilocybin, and a phosphorylated derivative of 4-HO-NMT (4-hydroxy-N-methyltryptamine).

As used herein, the terms “therapeutically effective amount” or “therapeutically effective dosage,” when referring to a drug or therapeutic agent, is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the terms “treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

### PHARMACEUTICAL COMPOSITIONS

The present disclosure provides pharmaceutical compositions comprising at least one deep eutectic solvent and one or more pharmaceutically active ingredients (APIs) extracted from a fungal biomass, e.g., psilocybin mushrooms. In some embodiments, the one or more APIs are selected group consisting of baeocystin, norbaeocystin, N,N-dimethyltryptamine, 5-hydroxytryptamine (serotonin), 5-hydroxytryptophan, psilocybin and psilocin. In some embodiments, the pharmaceutical compositions are free from additives, i.e., the pharmaceutical compositions comprise less than 5%, less than 4%, less than 3%, less than 2%, less than 1%, less than 0.5% by weight of any additives.

A deep eutectic solvent (“DES”) is a type of ionic liquid with special properties composed of a mixture which forms a eutectic with a melting point much lower than either of the individual components. Said another way, the term “deep ionic solvent” denotes an ionic solvent which is formed by a mixture of compounds that form an eutectic system with a melting point significantly lower than that of each of its individual components.

In general, a DES may be prepared by simply warming and stirring the components for a certain period of time, optionally with a successive drying step. For example, a DES may be prepared by a thermal treatment of two mixed components (see Abbott (2003) supra; Abbott, A. P., et al., ChemPhysChem 7 (2006) 803) or by a freeze-drying procedure (Gutierrez, M. C., et al., Langmuir 25 (2009) 5509; ibid Angew. Chem. Int. Ed. 49 (2010) 2158). By way of another example, DESs can be produced by mixing choline chloride (ChCl) with an organic hydrogen donor, e.g. an amine, amide, alcohol, or carboxylic acid (see e.g. Abbott, A. P., et al., J. Am. Chem. Soc. 126 (2004) 9142-9147; ibid. Chem. Commun. (2003) 70-71). By way of another example, choline chloride may be mixed with an aliphatic hydrogen bond donor comprising at least one thiol group and at least one hydroxy group or salt thereof (e.g., dithiothreitol, 2-mercaptoethanol, 4-mercapto-1-butanol, 1-mercapto-2-propanol or sodium 2-mercaptoethansulfonate) at elevated temperature (e.g. at 120° C.) in the required molar ratio with agitation for half an hour and cooling the heated combination afterwards (after the DES has been formed). To remove traces of water drying with phosphor pentoxide can be performed afterwards (e.g., for two weeks at 45° C.).

Non-limiting examples of liquid components which may be mixed to form a deep eutectic solvent include, but are not limited to, those listed below. Indeed, any two, three, or four of the liquid components set forth below may be mixed to form a deep eutectic solvent.
1,8-cineole (Eucalyptol)
1-butyl-3-methylimidazolium chloride
1-decyl-2,3 dimethylimidazoliumchloride
1-decyl-2,3-dimethylimidazolium chloride
1-decyl-3-methylimidazolium chloride
1-dodecyl-3-methylimidazolium chloride
1-ethyl-3-methylimidazolium chloride
1-octyl-2,3-dimethylimidazolium chloride
1-octyl-2,3-dimethylimidazoliumchloride
1-octyl-3-methylimidazolium chloride
Acetylcholine chloride
Arginine
Benzalkonium chloride
Benzyl-tributyl-ammonium-chloride
Benzyl-trimethyl-ammonium-chloride
benzyltriphenylphosphonium chloride
Benzyl-tripropyl-ammonium-chloride
Betaine
Betaine hydrochloride
D-(+)-glucose
Borneol
C₄mim(CF₃CO₂)₂N
C₄mimBF₄
C₄mimCl
Capric acid
Choline
Choline chloride
3,4-dihydroxybenzoic acid
N-methyl urea
Cholinium acetate
Cholinium hydroxide
Citric acid
Ethambutol
L-Arginine
Coenzyme Q
dimethylaminopropanol
d-limonene
Ethylimidazole
Ethylene Glycol
Glucose
Glycerol
Glycolic Acid
D-(-)-fructose
xylitol
Ibuprofen
Imipramine
Lactic acid
Levelunic Acid
Water
Lidocaine
L-proline
Malic acid
Menthol
Menthone
Methylimidazole
Methyltrioctylammonium chloride
octylic acid
Methyltriphenylphosphine bromide
Methyltriphenylphosphonium bromide
Myristic acid
N,N-diethylethanolammonium chloride
Nimesulide
dodecanoic acid
1-nonanoic acid
Oxalic Acid
p-cymene
Proline
tartaric acid
Tetrabutylammonium bromide
Tetrabutylammonium chloride
Tetraethylammonium p-toluene sulfonate
Tetraheptylammonium chloride
Tetraoctylammonium chloride
Tetrapropylammonium bromide
Thymol
Tributylmethylammonium chloride
Trioctylamine
Trioctylammonium bromide
Zinc chloride
ZnCl₂
β-alanine
ethanol
1-undecanol
1-dodecanol
3-hydroxycarboxylic acid
Acetamide
Glutamic acid
Acrylic acid
Diethylene glycol
Triethylene glycol
2,2,2-trifluoroacetamide
Phenol
2-furoic acid
Erythritol
Phenylacetic acid
S-(+)-mandelic acid
Urea
Butanediols
AlCl₃
Lauric acid
Limonene
Stearic acid
Geranate
1,1-dimethyl urea
1,3-butanediol
1,3-dimethyl urea
1,3-propanediol [1,3 PDO]
1,4-butanediol
1,6-hexanediol
1-methyl urea
2,2,2 Triflouracetamid
5,6,7,8-tetrahydro-5,5,8,8-tetramethylnaphtalen-2-ol
Acetic acid
Acetylsalicylic acid
Aconitic acid
Adipic acid
Ascorbic acid
Benzamide
Benzoic acid
Butanediol
Butyric acid
Caprylic acid
Chlorophenol
CrCl₃·6H₂O
D(+) Glucose
Diethanolamine
D-sorbitol
D-xylose
Ethanolamine
Formic acid
Fructose
Glutaric acid
Itaconic acid
L(+) tartaric acid
L-cysteine
Levulinic acid
L-methionine
L-serine
L-threonine
Maleic acid
Malonic acid
Maltose
Mandelic acid
Methacrylic acid
Methyldiethanolamine
MgCl₂·6H₂O
Octanoic acid [OCT]
phenylpropionic acid
Polyethylene glycol
Propanediol
Propionic acid
propylene glycol
P-toluene sulfonic acid
Resorcinol
Sorbitol
succinic acid
sucrose
Tartaric Acid
Tetraethylene glycol [PEG-4]
thiourea
Toluenesulfonic acid
tricarballylic acid
Triethanolamine
Valeric acid
Zinc bromide
Glutamine
Phenylalanine
D-(+)-maltose
L-arginine or ethambutol
1,4, butanediol
Dimethyl urea
Glycine
Sodium acetate
Trisodium citrate
L-alanine
Nicotinamide
Camphor
Prilocaine
Tetracaine
Dodecanol
Hexanoic acid
1,3-propanediol
Octanoic acid
Tetraethylene glycol
Nictonamide
Decanoic acid
Propionic acid
1,2-propanediol
Butanol
Decanol
Octanol
Hexanediol

Preferred liquid components which may be combined to form a deep eutectic solvent include, but are not limited to, choline oleate, choline hexanoate, choline geranate, choline malonate (choline disodium malonate), and urea-choline.

In some embodiments, the pharmaceutical compositions include deep eutectic solvent formed from two liquid components, e.g., from two different liquids or solvents. In some embodiments, a molar ratio of a first liquid component to a second liquid component ranges from 5:1 to 1:5. In some embodiments, a molar ratio of a first liquid component to a second liquid component ranges from 4:1 to 1:4. In some embodiments, a molar ratio of a first liquid component to a second liquid component ranges from 3:1 to 1:3. In some embodiments, a molar ratio of a first liquid component to a second liquid component ranges from 2:1 to 1:2. In some embodiments, a molar ratio of a first liquid component to a second liquid component ranges from 1:1 to 1:1. Exemplary molar ratios of a first liquid component to a second liquid component include, but are not limited to, 1:1, 1:2, 2:1, 1:3, 3:1, 2:3, 3:2, and ranges between these ratios.

Examples of a combination of first and second eutectic solvents and the ratios of each eutectic solvent relative to each other are provided in the Table below:

| | |
|---|---|
| Choline Chloride: Glucose | 1:1 |
| Choline Chloride: Fructose | 1:1 |
| Choline chloride: ethylene glycol | 1:2 |

| | |
|---|---|
| Choline chloride: 1,2-propanediol | 1:1 |
| Choline Chloride: Glycerol | 1:1 |
| Choline chloride: 1,4-butanediol | 1:5 |
| Choline chloride: citric acid | 1:1 |
| Choline Chloride: Malic Acid | 1:1 |
| Choline chloride: Geranic acid | 1:1 |
| Choline Chloride: Malonic Acid | 1:1 |
| Choline Chloride: Urea | 1:2 |
| Betaine: Glycerol | 1:2 |
| Betaine: Citric acid | 1:1 |
| Betaine: Malic acid | 1:1 |
| Proline: Glycerol | 2:5 |
| Lactic acid: glucose | 5:1 |
| Choline chloride: Geranic Acid | 1:2 |

In some embodiments, the pharmaceutical compositions include deep eutectic solvents formed from three or more liquid components. In some embodiments, a molar ratio of a first liquid component to a second liquid component to a third liquid component ranges from 5:1:5. In some embodiments, a molar ratio of a first liquid component to a second liquid component to a third liquid component ranges from 4:1:4. In some embodiments, a molar ratio of a first liquid component to a second liquid component to a third liquid component ranges from 3:1:3. In some embodiments, a molar ratio of a first liquid component to a second liquid component to a third liquid component ranges from 2:1:2. In some embodiments, a molar ratio of a first liquid component to a second liquid component to a third liquid component ranges from 1:1:1. Exemplary molar ratios of a first liquid component to a second liquid component to a third liquid component include, but are not limited to, 1:1:1, 1:2:1, 2:1:2, 1:3:1, 3:1:3, 2:3:2, 3:2:3, and ranges between these ratios.

Examples of a combination of first, second, and third eutectic solvents and the ratios of each eutectic solvent relative to each other are provided in the Table below:

| | |
|---|---|
| Choline Chloride: Malic Acid: Xylitol | 1:1:1 |
| Choline Chloride: Malic Acid: Proline | 1:1:1 |
| Choline Chloride: Urea: 1,2-Propanediol | 1:1:1 |

| | |
|---|---|
| Betaine: Malic Acid: Glucose | 1:1:1 |
| Betaine: Proline: Glucose | 1:1:1 |
| Betaine: Proline: Malic acid | 1:1:1 |
| Betaine: Proline: Sucrose | 1:1:1 |

Yet another example of a combination of three solvents is dichloroacetic acid : 1-menthol : and n-butanol in a ratio of 4:1:1. An example of a combination of four solvents is proline : malic acid : lactic acid : water in a ratio of 1:2:0.3:0.5.

### PHARMACEUTICALLY ACCEPTABLE FORMULATIONS

The present disclosure provides pharmaceutically acceptable formulations comprising (i) at least one deep eutectic solvent, (ii) one or more pharmaceutically active ingredients extracted from a fungal biomass, e.g., psilocybin mushrooms, and (iii) one or more pharmaceutically acceptable carriers or excipients. In some embodiments, the one or more APIs are selected group consisting of baeocystin, norbaeocystin, N,N-dimethyltryptamine, 5-hydroxytryptamine (serotonin), 5-hydroxytryptophan, psilocybin, psilocin, and any combination thereof. Non-limiting examples of additives include penetration or permeation enhancers, plasticizers, gelling agents, fluid carriers, oils, tackifiers, cohesion-promoting additives, stabilizers, fillers, colorants, dyes, UV-absorbing compounds, buffers, preservatives, emulsifiers, antimicrobial and/or antifungal agents, wetting agents, antioxidants and similar additives. As described herein, the pharmaceutically acceptable formulations may be applied directly to the skin of a subject in need of treatment thereof or as part of an external preparation for application to the skin.

### Penetration Enhancers or Permeation Enhancers

In some embodiments, the pharmaceutically acceptable formulations include one or more penetration or permeation enhancers. As used herein, a “penetration enhancer” or a “permeation enhancer” refers to a chemical that aids transport across the epithelium of the skin (stratum corneum), such as by altering the structure of the cellular membrane (transcellular route) and/or the tight junctions between cells (paracellular route) of the stratum corneum. In some embodiments, these penetration or permeation enhancers are intended to promote penetration of the active agent through the skin, and suitable enhancers include those described in U.S. Pat. No. 5,503,844, including monovalent, saturated and unsaturated aliphatic and cycloaliphatic alcohols having 6 to 12 carbon atoms such as cyclohexanol, lauryl alcohol, and the like; aliphatic and cycloaliphatic hydrocarbons such as mineral oil; cycloaliphatic and aromatic aldehydes and ketones such as cyclohexanone; N,N-di(lower alkyl) acetamides such as N,N-diethyl acetamide and N,N-dimethyl acetamide, N,N-dimethyl acetamide, N-(2-hydroxyethyl)acetamide and the like; aliphatic and cycloaliphatic esters such as isopropyl myristate and lauricidin; N,N-di(lower alkyl) sulfoxides such as decylmethyl sulfoxide; essential oils, nitrated aliphatics, aliphatic and cycloaliphatic hydrocarbons such as N-methyl-2- pyrrolidone and azone; salicylates, polyalkylene glycol silicates; aliphatic acids such as oleic acid and lauric acid, terpines such as cineole, surfactants such as sodium lauryl sulfate, siloxanes such as hexamethyl siloxane; mixtures of the above materials; and the like.

Exemplary cationic penetration enhancers (CPEs) include, but are not limited to cationic surfactants, cationic polymers (e.g., polylysine, polyethylene imine, polyarginine), fatty amines, and nitrogen-containing rings. Exemplary anionic penetration or permeation enhancers include, but are not limited to, anionic surfactants (e.g., sodium lauryl sulfate, sodium decyl sulfate, sodium octylsulfate), and salts of fatty acids. Examples of CPEs include, but are not limited to, the following:
Sodium lauryl sulfate
Sodium decyl sulfate
Sodium octyl sulfate
Sodium laureth sulfate
N-Lauryl sarcosinate
Cetyltrimethyl ammonium
bromide
Decyltrimethyl ammonium
bromide
Benzyldimethyl dodecyl
ammonium chloride
Myristyltrimethyl ammonium
chloride
Dodecyl pyridinium chloride
Decyldimethyl ammonio
propane sulfonate
Myristyldimethyl ammonio
propane sulfonate
Palmityldimethyl ammonio
propane sulfonate
ChemBetaine CAS
ChemBetaine Oleyl
Palmitoyl carnitine chloride
Sodium deoxycholate
Sodium glycocholate
Cholic acid
Hexanoic acid
Heptanoic acid
Sodium oleate
Urea
Lauryl amine
Caprolactam
Methyl pyrrolidone
Octyl pyrrolidone
Methyl piperazine
Phenyl piperazine

Exemplary penetration enhancers include methyl laurate, ethyl oleate, glycerol mono oleate, oleic acid, oleyl alcohol, isopropyl palmitate, isopropyl myristate, octyldodecanol, ω-pendadecalactone, cyclopendadecanone, propylene glycol monolaurate, eucalyptol, Ceraphyl 31, 1-dodecanol, transcutol P, triacetin, propylene glycol, dipropylene glycol, butylene glycol, ethanol, octanol, limonene, sorbitan monooleate, n-alkylphenol ether ethoxylates, n-alkyl ether ethoxylates, and mixtures thereof.

### Antioxidants

The term “antioxidant” is used herein includes any compound or combination of compounds that prevent or slow down oxidation of components caused by the damaging reactive oxygen species (ROS). Any of the known antioxidants may be used, including but not limited to tocopherols, phospholipids (PL), phytosterols, phycocyanin, vitamins E, A and C, betacarotene, coenzyme Q10, fatty acids omega-3, omega-6 and w-9, phytoantioxidants such as polyphenols, terpenes as butylated hydroxyanisole (BHA), butylated hydroxytoluene (BHT), propyl gallate, lecithin, sesamin, sesamol, sesamolin, α-tocopherol, γ-tocopherol, salicylic acid, ascorbic acid, ascorbyl palmitate, fumaric acid, malic acid, sodium ascorbate and sodium meta-bisulphite, as well as chelating agents such as disodium EDTA. Pharmaceutically acceptable nutraceutical dietary supplements may also be employed as anti-oxidants including plants, alga, and lichen and may include one or more extracts of honeybee propolis, red clover, soybean, caper, almond, milk thistle, green tea, pomegranate, orange red, grape seed, bilberry, fo-ti root, ginseng, English ivy, red algae, brown algae, green algae and lichens.

### Binders

Examples of binders that may be included in the compositions of the present disclosure include conventional hydrogels formed using water-soluble or water-insoluble gums or resins, with or without known cross-linking agents. The gums or resins include agarose, alginates, alkyl and hydroxyalkyl celluloses, such as hydroxyethyl cellulose and hydroxypropyl cellulose, amylopectin, arabinogalactin, carboxymethyl cellulose, carrageenan, eucheuma, ucoidan, furcellaran, gelatin, guar gum, gum agar, gum arabic, gum ghatti, gum karaya, gum tragacanth, pypenia, keratin laminaran, locust bean gum, pectin, polyacrylamide, poly(acrylic)acid and homologs, polyethylene glycol, poly(ethylene oxide), poly(hydroxyalkyl) methacrylate, polyvinyl alcohol, polyvinylpyrrolidone, propylene glycol alginate, starch and modified analogs, tamarind gum, N-vinyl lactam polysaccharides and xanthan gum. In addition, such hydrogels can be formed by the copolymerization and cross-linking of both hydrophilic and hydrophobic monomers, such as hydroxy-alkyl esters of acrylic acid and methacrylamide, n-vinyl-1-pyrrolidone, alkyl acrylates and methacrylates, vinyl acetate, acrylonitrile and styrene. Other binders suitable for use with the present disclosure include veegum, higher molecular weight polyglycols, and the like.

### Buffers

Suitable buffers are selected from of 2,2-Bis(hydroxymethyl)-2,2′,2″-nitrilotriethanol (“Bis-Tris”), 1,4-Piperazinediethanesulfonic acid (“PIPES”), β-Hydroxy-4-morpholinepropanesulfonicacid (“MOPSO”), N,N-Bis(2-hydroxyethyl)-2-aminoethanesulfonic acid (“BES”), 3-(N-Morpholino)propanesulfonic acid (“MOPS”), 4-(2-Hydroxyethyl)piperazine-1-ethanesulfonic acid (“HEPES”), 3-(N,N-Bis[2-hydroxyethyl]amino)-2-hydroxypropanesulfonic acid (“DIPSO”), 4-(N-Morpholino) butanesulfonic acid (“MOBS”), 4-(2-Hydroxyethyl) piperazine-1-(2-hydroxypropanesulfonic acid) (“HEPPSO”), piperazine-1,4-bis(2-hydroxypropanesulfonicacid) dihydrate (“POPSO”), 4-(2-Hydroxyethyl)-1-piperazinepropanesulfonic acid (“EPPS”), Imidazoleimidazole, and Maleatemaleate.

### Carriers

In some embodiments, the carrier is an oil-based carrier, such as a hydrocarbon-based oil.” In some embodiments, a “hydrocarbon-based oil” refers to an oil predominantly containing carbon and hydrogen atoms and especially alkyl or alkenyl chains, for instance alkanes or alkenes, but also an oil containing, in addition to hydrogen and carbon atoms, oxygen atoms in the form of an ether, ester, alcohol or carboxylic acid function. In some embodiments, the hydrocarbon-based oils include, but are not limited to, liquid paraffin or liquid petroleum jelly, mink oil, turtle oil, soybean oil, perhydrosqualene, sweet almond oil, beauty-leaf oil, palm oil, grapeseed oil, sesame oil, corn oil, parleam oil, arara oil, rapeseed oil, sunflower oil, cotton oil, apricot oil, castor oil, avocado oil, jojoba oil, olive oil or cereal germ oil; esters of lanolic acid, of oleic acid, of lauric acid and of stearic acid, fatty esters, such as isopropyl myristate, isopropyl palmitate, butyl stearate, hexyl laurate, diisopropyl adipate, isononyl isononanoate, 2-ethyhexyl palmitate, 2-hexyldecyl laurate, 2-octyldecyl palmitate, 2-octyldodecyl myristate or lactate, bis(2-ethylhexyl) succinate, diisostearyl malate, glyceryl triisostearate or diglyceryl triisostearate; higher fatty alcohols containing at least 12 carbon atoms, such as stearyl alcohol or oleyl alcohol, linoleyl alcohol linolenyl alcohol, isostearyl alcohol or octyldodecanol.

In other embodiments, the oil-based carrier is selected from a botanical and/or an essential oil, such as Lavandula angustifolia (lavender) oil, pelargonium graveolens flower oil, citrus aurantium dulcis (orange) peel oil, Rosmarinus officinalis (rosemary) leaf oil, menthe viridis (spearmint) leaf oil, citrus aurantifolia (lime) oil, melaleuca alternifolia (tea tree) leaf oil, citrus grandis (grapefruit) peel oil, citrus medica limonum (lemon) peel oil, rose flower oil, eucalyptus globulus leaf oil, and combinations thereof.

In yet other embodiments, the oil-based carrier includes one or more silicone-based oils, for example poly(C₁-C20) alkylsiloxanes, such as polyalkylniethylsiloxanes and further such as volatile and non-volatile, linear and cyclic polydimethylsiloxanes (PDMSs), such as cyclotetradimethylsiloxane, cyclopentadimethylsiloxane and cyclohexadimethylsiloxane; silicones modified with aliphatic and/or aromatic groups, which may be fluorinated, or with functional groups such as hydroxyl, thiol and/or amine groups; phenylsilicone oils such as polyphenylmethylsiloxanes or phenyltrimethicones.

### Antimicrobial and Antifungal Actives

Examples of antimicrobial and antifungal actives include β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate and clotrimazole.

### Preservatives

Suitable preservatives in the composition of the present invention may include, but not limited to, phenoxyethanol, alkyl para-hydroxybenzoates, wherein the alkyl radical has from 1, 2, 3, 4, 5 or 6 carbon atoms and preferably from 1 to 4 carbon atoms e.g., methyl parahydroxybenzoate(methylparaben), ethyl para-hydroxybenzoate(ethylparaben), propyl parahydroxybenzoate(propylparaben), butyl para-hydroxybenzoate(butylparaben), isobutyl parahydroxybenzoate(isobutylparaben), and their mixtures.

### Emulsifiers

The compositions of the present disclosure may also include various emulsifiers. In the final product compositions of the present disclosure, emulsifiers may be included in the amount of up to about 10%, preferably, in the amount of from about 0.5% to about 5% by weight of the composition. The examples of suitable emulsifiers include stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, polyethyleneglycols, polypropyleneglyocis, and mixtures thereof.

In some embodiments, the compositions or formulations of the present disclosure may be in the form of an emulsion which may contain surfactants or a mixture thereof. Suitable surfactants for use in a composition of the present invention in the form of an emulsion include anionic, nonionic, amphoteric and cationic surfactants. See, e.g., Encyclopedia of Chemical Technology, KIRK-OTHMER, volume 22, pp. 333-432, 3rd edition, 1979, Wiley, for the definition of the properties and (emulsifying) functions of the surfactants, in particular pp. 347-377 of this publication regarding anionic and nonionic surfactants.

### TRANSDERMAL ADMINISTRATION OF THE PHARMACEUTICAL COMPOSITIONSS OR PHARMACEUTICALLY ACCEPTABLE FORMULATIONS

Dosage forms for topical administration include, but are not limited to, ointments, creams, emulsions, lotions, gels, sunscreens and agents that favor penetration within the epidermis.

In some embodiments, the pharmaceutical compositions or the pharmaceutically acceptable formulations of the present disclosure may be administered transdermally, such as to a subject (e.g., a mammalian subject, a human patient) in need of treatment thereof. In some embodiments, the pharmaceutical composition or the pharmaceutically acceptable formulations of the present disclosure may be applied directly to the skin of a subject in need of treatment thereof, i.e., the pharmaceutical compositions or the pharmaceutically acceptable formulations including the one or more APIs, the one or more eutectic solvents, and/or one or more additives may be applied directly to the skin. For example, the pharmaceutical composition or the pharmaceutically acceptable formulations of the present disclosure may be applied by directly contacting the skin of the subject with a therapeutically effective amount of the pharmaceutically acceptable formulation; and then optionally covering the therapeutically effective amount of the pharmaceutically acceptable formulation applied to the skin. In some embodiments, the pharmaceutical composition or the pharmaceutically acceptable formulation is administered through the skin located at various parts of the body but preferably at the hips, the ventral and lateral parts of the thorax, the thighs and the ventral side of the arms.

Alternatively, the pharmaceutical compositions or the pharmaceutically acceptable formulations of the present disclosure may be included with any conventional dosage form or external preparation, such as such as a tape, a patch, a cataplasm, an ointment, a cream, a lotion, a liquid, a gel or the like. In some embodiments, external preparations in these dosage forms can be prepared by usual methods using a conventional adhesive, base or the like. For example, the external preparations may be prepared according to the methods described by “Keihi Tekiyou Seizai Kaihatu Manual (A Development Manual of Formulations for Transdermal Application)” edited by Mitsuo MATSUMOTO (1985), JP-2651616, WO96/12465, JP-A-9-278651.

In some embodiments, the pharmaceutical compositions or the pharmaceutically acceptable formulations of the present disclosure are included within a tape or a patch for application to the skin of the subject. In some embodiments, the tape or patch includes an adhesive layer including any of the compositions of the present disclosure, and a support (e.g., a backing layer) that supports the adhesive layer. In some embodiments, the tape or patch may include a release liner that prevent or mitigates exposure of the adhesive layer before use and that can be easily removed from the adhesive layer at the time of use, i.e., at the time of administration of the tape or patch to the skin of a subject in need of treatment thereof.

In some embodiments, the pharmaceutical compositions or the pharmaceutically acceptable formulations of the present disclosure are included within a reservoir layer of any tape or patch. Said another way, instead of the pharmaceutical compositions or the pharmaceutically acceptable formulations of the present disclosure being mixed with or incorporated within any adhesive layer, the pharmaceutical compositions or the pharmaceutically acceptable formulations of the present disclosure are included with a separate reservoir layer. In some embodiments, the reservoir layer is sandwiched between a support or backing layer, which is impermeable to the one or more APIs included within the pharmaceutical compositions or the pharmaceutically acceptable formulations of the present disclosure, and an in-line contact adhesive layer. Such a tape or patch may further include a release liner as described above.

In some embodiments, the pharmaceutical compositions or the pharmaceutically acceptable formulations of the present disclosure may be mixed or dispersed within one or more adhesives. As noted above, in some embodiments, the adhesives may be incorporated within a patch or a tape for application to the skin of a subject in need of treatment thereof.

Any adhesive may be used to form the adhesive layer. In some embodiments, an adhesive for a tape or a patch includes, for example, an acrylic adhesive, a rubber adhesive, a silicon adhesive or the like. In some embodiments, an acrylic adhesive includes, for example, a (co)polymer of alkyl (meta)acrylate (as a main component). The (co)polymer may be either a copolymer of two or more kinds of alkyl (meta)acrylate or a copolymer of an alkyl (meta)acrylate with a functional monomer which is copolymerizable with alkyl (meta)acrylate. The alkyl acrylate includes, for example, acrylates which are esterified by a straight chain or branched chain alkyl having 1 to 18 carbon atoms, specifically methyl (meta)acrylate, butyl (meta)acrylate, hexyl (meta)acrylate, octyl (meta)acrylate, nonyl (meta)acrylate, decyl (meta)acrylate or the like. The functional monomer includes, for example, a monomer having a hydroxyl group (e.g. hydroxyethyl (meta)acrylate), a monomer having a carboxyl group (e.g. butyl maleate or crotonic acid), a monomer having an amide group (e.g. (meta)acrylamide), a monomer having an amino group (e.g. dimethylaminoacrylic acid esters), a monomer having a pyrrolidone ring (e.g. N-vinyl-2-pyrrolidone), or the like.

In some embodiments, the adhesive is polyisobutylene. In some embodiments, the polyisobutylene may be high molecular weight polyisobutylene, low molecular weight polyisobutylene, or a mixture thereof. In some embodiments, the high molecular weight polyisobutylene may have an average molecular weight of 500,000 to 1.5 million, or from 750,000 to 1.2 million. In some embodiments, the low molecular weight polyisobutylene may have an average molecular weight of 40,000 to 85,000. Suitable polyisobutylene adhesives include Oppanol B80 (a high molecular weight PIB), Oppanol B100 (a high molecular weight PIB), Oppanol B12 (a low molecular weight PIB), and Duro-Tak 87-613A (a mixture of high and low molecular weight PIBs). For adhesives using a mixture of high and low molecular weight PIBs, the ratio of high molecular weight to low molecular weight PIB may range from 0.5:1 to 1.5:1; 0.6:1 to 1.1:1; or about 0.8:1.

In some embodiments, an additive which is added to a tape or a patch includes α-terpineol, isopropyl myristate, 1-menthol, lauric acid, lauryl alcohol, crotamiton, diethyl sebacate, N-methyl-2-pyrrolidone, Azone (Registered Trademark) or the like.

In some embodiments, a rubber adhesive includes, for example, a natural rubber, a polyisopropylene rubber, a polyisobutylene rubber, a styrene-isoprene-styrene block copolymer or a styrene-butadiene-styrene block copolymer, wherein the main component is a rubber elastomer.

In some embodiments, a silicon adhesive includes, for example, a polydimethylsiloxane or a diphenylsiloxane wherein the main component is a silicon rubber.

In some embodiments, the pharmaceutical compositions or the pharmaceutically acceptable formulations of the present disclosure are provided as a base for an ointment, a cream, a lotion, a liquid, or a gel preparation. In some embodiments, an ointment or a cream preparation includes the base and, for example, a fatty oil, a lanolin, a petrolatum, a paraffin, a plastibase, glycols, a higher fatty acid, a higher alcohol or the like. A stabilizing agent, an antiseptic agent, an emulsifying agent, a suspending agent or the like may be optionally added to the ointment or the cream preparation. In some embodiments, a lotion preparation includes the base and for example, ethanol, glycerine, glycol or the like. In some embodiments, a liquid preparation includes the base and, for example, ethanol, water, glycol or the like. In some embodiments, a gel preparation includes the base and, for example, a product obtained by gelatinizing liquid fats and oils by a gelling agent for an oily gel preparation, or a gelling agent such as a carboxymethyl polymer, a hydroxypropylcellulose or a polyvinyl alcohol for an aqueous gel preparation.

In some embodiments, an amount of the one or more APIs included within the pharmaceutical compositions or the pharmaceutically acceptable formulations of the present disclosure varies depending on the age, sex, weight, body-surface-area, etc. of the subject in need of treatment thereof. In some embodiments, the one or more APIs included within the pharmaceutical compositions or the pharmaceutically acceptable formulations of the present disclosure ranges from about 0.01 mg to 1.0 g/day in adults. In some embodiments, it is preferable that a blood level to be maintained through administration of the one or more APIs included within the pharmaceutical compositions or the pharmaceutically acceptable formulations of the present disclosure be within a maximum blood level attained through oral administration of the one or more APIs, in view of current clinical use of the one or more APIs (and, of course, in view of any reported side-effects). In some embodiments, the pharmaceutical compositions or the pharmaceutically acceptable formulations are administered to the subject in need of treatment thereof for a time period ranging from between about 1 hour to about 72 hours.

In some embodiments, the pharmaceutical compositions or the pharmaceutically acceptable formulations disclosed herein may be administered in a combination therapy, i.e., either simultaneously in single or separate dosage forms or in separate dosage forms within hours or days of each other. Examples of compounds/drugs used in such combination therapies include without limitation, chemotherapeutic agents, immunosuppressive agents, immunostimulatory, anti-pyretic, cytokines, opioids, cytokines, cytotoxic agents, nucleolytic compounds, radioactive isotopes, receptors, pro-drug activating enzymes, which may be naturally occurring or produced by recombinant methods, anti-inflammatory agents, antibiotics, protease inhibitors, growth factors, osteo-inductive factors and the like.

In some embodiments, an effective amount of the pharmaceutical compositions or the pharmaceutically acceptable formulations of the present disclosure may be administered to a subject in need of treatment for the treatment of anxiety, depression, and other mental health conditions. In some embodiments the mental health condition is selected from the group consisting of attention deficit hyperactivity disorder, depressive personality disorder, dysphoric disorder, obsessive-compulsive disorder, dysthymia double depression, atypical depression, melancholid depression, psychotic major depression, catatonic depression, postpartum depression, premenstrual seasonal affective disorder, depressive disorder not otherwise specified, recurrent brief depression, and minor depressive disorder.

### STABILITY AND PHARMACOKINETICS

The pharmaceutical compositions or the pharmaceutically acceptable formulations disclosed herein provide several advantages, including but not limited to, increased shelf stability, increased bioavailability, and increased bioactivity.

In some embodiments, the pharmaceutical compositions or the pharmaceutically acceptable formulations disclosed herein do not degrade to an unacceptable extent such that the composition has a shelf-life of at least about 2 years. As previously mentioned, this means that any APIs within the pharmaceutical compositions or the pharmaceutically acceptable formulations disclosed herein remain within 90-110% of its initial amount in the dosage form during the desired (e.g., labeled) shelf-life of the dosage form (e.g., a minimum of 2 years after the date of manufacture of the dosage form).

The pharmaceutical compositions or the pharmaceutically acceptable formulations disclosed herein may have a shelf half-life of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 240, 270, 300, 330, or 360 days. In some embodiments, the pharmaceutical compositions or the pharmaceutically acceptable formulations disclosed herein may have a shelf half-life of at least about 1, 2, 3, 4, or 5 years.

### METHODS OF EXTRACTING ONE OR MORE APIS FROM MUSHROOMS

A first step encompasses obtaining a fungal biomass. In some embodiments, the obtained fungal biomass comprises psilocybin mushrooms.

Next, the obtained fungal biomass is optionally pre-processed. In some embodiments, pre-processing comprises cutting, shearing, mincing, or slicing the mushrooms, preferably without bruising the mushrooms.

Next, the processed one or more mushrooms are transferred to a deep eutectic solvent, including any of those described herein, to provide a mixed solution.

Subsequently, the obtained fungal biomass or the pre-processed fungal biomass is transferred to a deep eutectic solvent, including any of the deep eutectic solvents disclosed herein, to provide a mixture. In some embodiments, the mixture of the fungal biomass and the deep eutectic solvent is heated, such as heated to room temperature or greater. In some embodiments, the mixture is heated to at least 25° C., at least 26° C., at least 27° C., at least 28° C., at least 29° C., at least 30° C., at least 31° C., at least 32° C., at least 33° C., at least 34° C., at least 35° C., at least 36° C., at least 37° C., at least 38° C., at least 39° C., at least 40° C., etc. In some embodiments, the extraction is allowed to take place over a period of time ranging from between about 1 minute to about 5000 minutes. In some embodiments, the extraction takes place for at least 1 minute, at least 5 minutes, at least 10 minutes, at least 20 minutes, at least 60 minutes, at least 120 minutes, at least 240 minutes, at least 480 minutes, etc.

In some embodiments, acoustic waves are introduced into the mixed solution. In some embodiments, the acoustic waves are ultrasonic waves. In some embodiments, the ultrasound frequency introduced ranges from about 100 KHz to about 50 MHz. In some embodiments, a frequency in the range of about 0.1 to about 1 MHz can be used but only at a low power range of about 0.1 to about 25 W/cm² or else cavitation will occur. In some embodiments, a frequency of about 1 MHz to 10 about MHz is believed to be less destructive than the lower frequencies and allows the use of higher power (e.g., about 20 to about 100 W/cm²) which helps chemicals penetrate into the mushroom.

Ultrasound and temperature control can also be used in the processing steps. Microcavitations caused by ultrasound irradiation may lead to instant alternative high pressure and low pressure in the mixed treatment solution and the fungal biomass contained therein. It is believed that this effect leads to emulsification and degassing in any fungal biomass. Ultrasound also produces strong convection in the solution under irradiation, leading to good solution circulation. No stirring is necessary when ultrasound is applied.

By way of example, in some embodiments, the deep eutectic solvent and mushroom combination can be extracted by placing the mixture into a beaker and into an ultrasonic bath with water heated to 30° C. In some embodiments of this example, the mixture is sonicated for at least 10 minutes.

Following mixing, the combination is allowed to cool to room temperature. In some embodiments, the cooling to room temperature is passive. In other embodiments, the combination is actively cooled.

In some embodiments, any solids present within the mixture are removed, and the remaining liquid is transferred to another vessel. In some embodiments, the mixture is filtered to provide a pharmaceutical composition comprising the one or more APIs found in the fungal biomass and the deep eutectic solvent. In some embodiments, the mixture is centrifuged. For example, in some embodiments the mixture is centrifuged to force the solids to the bottom of the centrifuge tube such that the liquid contents may subsequently be decanted off. In one example, the mixture of mushroom and DES is vacuum filtered using a paper filter in a Buchner funnel fitted to a side arm Erlenmeyer flask.

### EXAMPLES

### Example 1

Into a 25 ml test tube was placed a 200 mg sample of Psilocybin containing biomass was mixed into 10 ml of Choline Geranate 1:2 (CAGE). After vortexing briefly, the mushroom material was extracted by placing it into an ultrasonic bath with a water temperature of 30° C. and sonicated for 10 min. The test tube was centrifuged at 10,000 rpm for 5 minutes. The liquid supernatant was withdrawn and further processed for transdermal application.

The transdermal application can take the form of the CAGE material directly to the skin, it can take the form of mixing into a gel with a 60% CAGE 1:2 w/w blend with propylene glycol as an excipient and cosolvent. In one application the CAGE material post-extraction is applied to a patch that is adhered to the dermis. In one application, the CAGE material post-extraction is applied to a patch with iontophoresis capabilities. Experimentally, CAGE 1:2 has a relatively high viscosity and can be blended with cosolvents for improved dermal application.

### Example 2

In another example, the fungal biomass can be blended into the selected deep eutectic solvents (DESs). This volume of liquid material can be heated and sonicated. The biomass can be removed by filtration or centrifugation.

### Example 3

In another example, fungal biomass can be extracted with water as a solvent and blended with the selected deep eutectic solvent to extract from the aqueous portion. This mixture is allowed to separate the phases for 24 hours so that complete separation of the two phases and full equilibration of biomolecules between the two phases was attained.

All of the U.S. Pats, U.S. Pat. application publications, U.S. Pat. applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

### ADDITIONAL EMBODIMENTS

Additional Embodiment 1 A pharmaceutical composition comprising a deep eutectic solvent and one or more APIs derived from a fungal biomass.

Additional Embodiment 2 The pharmaceutical composition of additional embodiment 1, wherein the fungal biomass comprises psilocybin mushrooms.

Additional Embodiment 3 The pharmaceutical composition of additional embodiment 1, wherein the one or more APIs include at least one serotonin agonist.

Additional Embodiment 4 The pharmaceutical composition of additional embodiment 1, wherein the one or more APIs are selected from the group consisting of include baeocystin, norbaeocystin, N,N-dimethyltryptamine, 5-hydroxytryptamine (serotonin), 5-hydroxytryptophan, psilocybin and psilocin.

Additional Embodiment 5 The pharmaceutical composition of any one of additional embodiments 1 - 4, wherein the deep eutectic solvent comprises a first liquid component and a second liquid component.

Additional Embodiment 6 The pharmaceutical composition of additional embodiment 5, wherein the first liquid component is choline chloride.

Additional Embodiment 7 The pharmaceutical composition of additional embodiment 6, wherein the second liquid component is selected from the group consisting of a sugar, an allylene glycol, a diol, and an acid.

Additional Embodiment 8 The pharmaceutical composition of additional embodiment 5, wherein a ratio of the first liquid component to the second liquid component ranges from 5:1 to 1:5.

Additional Embodiment 9 The pharmaceutical composition of additional embodiment 8, wherein the ratio ranges from 4:1 to 1:4.

Additional Embodiment 10 The pharmaceutical composition of additional embodiment 8, wherein the ratio ranges from 3:1 to 1:3.

Additional Embodiment 11 The pharmaceutical composition of additional embodiment 8, wherein the ratio ranges from 2:1 to 1:2.

Additional Embodiment 12 The pharmaceutical composition of additional embodiment 8, wherein the ratio is about 1 to about 1.

Additional Embodiment 13 The pharmaceutical composition of additional embodiment 5, wherein the deep eutectic solvent further comprises a third liquid component.

Additional Embodiment 14 The pharmaceutical composition of additional embodiment 13, wherein the third liquid component is selected from the group consisting of xylitol or proline.

Additional Embodiment 15 The pharmaceutical composition of any one of additional embodiments 1 - 4, wherein the deep eutectic solvent is selected from the group consisting of a mixture of Choline Chloride and Glucose, a mixture of Choline Chloride and Fructose; a mixture of Choline chloride and ethylene glycol; a mixture of Choline chloride and 1,2-propanediol; a mixture of Choline Chloride and Glycerol; a mixture of Choline chloride and 1,4-butanediol; a mixture of Choline chloride and citric acid; a mixture of Choline Chloride and Malic Acid; a mixture of Choline chloride and Geranic acid; a mixture of Choline Chloride and Malonic Acid; a mixture of Choline Chloride and Urea; a mixture of Betaine and Glycerol; a mixture of Betaine and Citric acid; a mixture of Betaine and Malic acid; a mixture of Proline and Glycerol; a mixture of Lactic acid and glucose; and a mixture of and Choline chloride and Geranic Acid.

Additional Embodiment 16 The pharmaceutical composition of any one of additional embodiments 1 - 4, wherein the deep eutectic solvent is selected from the group consisting of a mixture of Choline Chloride, Malic Acid, and Xylitol; a mixture of Choline Chloride, Malic Acid, and Proline; and a mixture of Choline Chloride, Urea, and 1,2-Propanediol.

Additional Embodiment 17 The pharmaceutical composition of any one of additional embodiments 1 - 4, wherein the deep eutectic solvent is selected from the group consisting of a mixture of Betaine, Malic Acid, and Glucose; a mixture of Betaine, Proline, and Glucose; a mixture of Betaine, Proline, and Malic acid; and a mixture of Betaine, Proline, and Sucrose.

Additional Embodiment 18 The pharmaceutical composition of any one of additional embodiments 1 - 4, wherein the deep eutectic solvent comprises a mixture of dichloroacetic acid, 1-menthol, and n-butanol.

Additional Embodiment 19 The pharmaceutical composition of any one of additional embodiments 1 - 4, wherein the deep eutectic solvent comprises a mixture of proline, malic acid, lactic acid, and water.

Additional Embodiment 20 The pharmaceutical composition of any one of additional embodiments 1 - 19, wherein the pharmaceutical composition further comprises one or more pharmaceutically acceptable carriers or excipients.

Additional Embodiment 21 The pharmaceutical composition of additional embodiment 20, wherein the one or more pharmaceutically acceptable carriers or excipients comprise penetration enhancers or permeation enhancers.

Additional Embodiment 22 The pharmaceutical composition of additional embodiment 21, wherein the penetration enhancers or permeation enhancers are selected from the group consisting of methyl laurate, ethyl oleate, glycerol mono oleate, oleic acid, oleyl alcohol, isopropyl palmitate, isopropyl myristate, octyldodecanol, ω-pendadecalactone, cyclopendadecanone, propylene glycol monolaurate, eucalyptol, Ceraphyl 31, 1-dodecanol, transcutol P, triacetin, propylene glycol, dipropylene glycol, butylene glycol, ethanol, octanol, limonene, sorbitan monooleate, n-alkylphenol ether ethoxylates, n-alkyl ether ethoxylates, and mixtures thereof.

Additional Embodiment 23 A method of treating a human subject comprising a therapeutically effective amount of the pharmaceutical composition of any one of additional embodiments 1 - 22 to the human subject in need of treatment thereof.

Additional Embodiment 24 The method of additional embodiment 23, wherein the administration is transdermal.

Additional Embodiment 25 A transdermal delivery system comprising (i) an adhesive layer including the pharmaceutical composition of any one of additional embodiments 1 - 22 blended within an adhesive material, and (ii) a backing layer.

Additional Embodiment 26 The transdermal delivery system of additional embodiment 25, further comprising a release liner.

Additional Embodiment 27 A monolithic suspension-blend transdermal patch comprising: (a) a backing layer having inner and outer surfaces and being substantially impervious to the pharmaceutical composition of any one of additional embodiments 1 - 22; (b) a first adhesive layer having a first surface covering at least a portion of the inner surface of the backing layer, the adhesive layer comprising a uniform suspension of a therapeutically-effective amount of the pharmaceutical composition in an adhesive; (c) a removable release liner in contact with a second surface of the adhesive layer.

Additional Embodiment 28 The monolithic suspension-blend transdermal patch of additional embodiment 27, wherein the adhesive comprises a pressure sensitive adhesive.

Additional Embodiment 29 The monolithic suspension-blend transdermal patch of additional embodiment 27, wherein the adhesive layer comprises between about 5% to about 35% of the pharmaceutical composition of any one of additional embodiments 1 - 22.

Additional Embodiment 30 The monolithic suspension-blend transdermal patch of additional embodiment 27, wherein the adhesive is polyisobutylene.

Additional Embodiment 31 A cream or ointment comprising (i) the pharmaceutical composition of any one of additional embodiments 1 - 22; and (ii) a fatty oil, a lanolin, a petrolatum, a paraffin, a plastibase, one or more glycols, a higher fatty acid, a higher alcohol, or any combination thereof.

Additional Embodiment 32 A lotion comprising (i) the pharmaceutical composition of any one of additional embodiments 1 - 22; and (ii) ethanol, glycerine, glycol, or any combination thereof.

Additional Embodiment 33 A gel comprising (i) the pharmaceutical composition of any one of additional embodiments 1 - 22; and (ii) a product obtained by gelatinizing liquid fats and oils by a gelling agent for an oily gel preparation, or a gelling agent such as a carboxymethyl polymer, a hydroxypropylcellulose or a polyvinyl alcohol for an aqueous gel preparation.

Additional Embodiment 34 A method of treating a patient suffering from anxiety, depression, and/or other mental health conditions comprising contacting the skin of the patient in need of treatment thereof with any of the articles of additional embodiments 25 - 33.

Additional Embodiment 35 A mixture comprising (i) a deep eutectic solvent, and (ii) a fungal biomass which has been cut, sheared, minced, or sliced.

Additional Embodiment 36 The method of additional embodiment 35, wherein the deep eutectic solvent is selected from the group consisting of a mixture of Choline Chloride and Glucose, a mixture of Choline Chloride and Fructose; a mixture of Choline chloride and ethylene glycol; a mixture of Choline chloride and 1,2-propanediol; a mixture of Choline Chloride and Glycerol; a mixture of Choline chloride and 1,4-butanediol; a mixture of Choline chloride and citric acid; a mixture of Choline Chloride and Malic Acid; a mixture of Choline chloride and Geranic acid; a mixture of Choline Chloride and Malonic Acid; a mixture of Choline Chloride and Urea; a mixture of Betaine and Glycerol; a mixture of Betaine and Citric acid; a mixture of Betaine and Malic acid; a mixture of Proline and Glycerol; a mixture of Lactic acid and glucose; and a mixture of and Choline chloride and Geranic Acid.

Additional Embodiment 37 A liquid extracted from or filtered from the mixture of any one of additional embodiment 35 - 36, wherein the liquid includes the deep eutectic solvent and one or more APIs derived from the fungal biomass.

Additional Embodiment 38 The liquid of additional embodiment 37, wherein the one or more APIs are selected from the group consisting of include baeocystin, norbaeocystin, N,N-dimethyltryptamine, 5-hydroxytryptamine (serotonin), 5-hydroxytryptophan, psilocybin and psilocin.

Additional Embodiment 39 A method of treating a patient suffering from anxiety, depression, and/or other mental health conditions comprising contacting the skin of the patient in need of treatment thereof with the liquid of additional embodiment 36.

Additional Embodiment 40 An adhesive blend comprising the liquid of any one of additional embodiments 35 - 36 and an adhesive.

1. A pharmaceutical composition comprising a deep eutectic solvent and one or more APIs derived from a fungal biomass.

2. The pharmaceutical composition of claim 1, wherein the fungal biomass comprises psilocybin mushrooms.

3. The pharmaceutical composition of claim 1, wherein the one or more APIs include at least one serotonin agonist.

4. The pharmaceutical composition of claim 1, wherein the one or more APIs are selected from the group consisting of include baeocystin, norbaeocystin, N,N-dimethyltryptamine, 5-hydroxytryptamine (serotonin), 5-hydroxytryptophan, psilocybin and psilocin.

5. The pharmaceutical composition of claim 1, wherein the deep eutectic solvent comprises a first liquid component and a second liquid component.

6. The pharmaceutical composition of claim 5, wherein the first liquid component is choline chloride.

7. The pharmaceutical composition of claim 6, wherein the second liquid component is selected from the group consisting of a sugar, an allylene glycol, a diol, and an acid.

8. The pharmaceutical composition of claim 5, wherein a ratio of the first liquid component to the second liquid component ranges from 5:1 to 1:5.

9. The pharmaceutical composition of claim 8, wherein the ratio ranges from 4:1 to 1:4.

10. The pharmaceutical composition of claim 5, wherein the deep eutectic solvent further comprises a third liquid component.

11. The pharmaceutical composition of claim 10, wherein the third liquid component is selected from the group consisting of xylitol or proline.

12. The pharmaceutical composition of claim 1, wherein the deep eutectic solvent is selected from the group consisting of a mixture of Choline Chloride and Glucose, a mixture of Choline Chloride and Fructose; a mixture of Choline chloride and ethylene glycol; a mixture of Choline chloride and 1,2-propanediol; a mixture of Choline Chloride and Glycerol; a mixture of Choline chloride and 1,4-butanediol; a mixture of Choline chloride and citric acid; a mixture of Choline Chloride and Malic Acid; a mixture of Choline chloride and Geranic acid; a mixture of Choline Chloride and Malonic Acid; a mixture of Choline Chloride and Urea; a mixture of Betaine and Glycerol; a mixture of Betaine and Citric acid; a mixture of Betaine and Malic acid; a mixture of Proline and Glycerol; a mixture of Lactic acid and glucose; and a mixture of and Choline chloride and Geranic Acid.

13. The pharmaceutical composition of claim 1, wherein the pharmaceutical composition further comprises one or more penetration enhancers selected from the group consisting of methyl laurate, ethyl oleate, glycerol mono oleate, oleic acid, oleyl alcohol, isopropyl palmitate, isopropyl myristate, octyldodecanol, ω-pendadecalactone, cyclopendadecanone, propylene glycol monolaurate, eucalyptol, Ceraphyl 31, 1-dodecanol, transcutol P, triacetin, propylene glycol, dipropylene glycol, butylene glycol, ethanol, octanol, limonene, sorbitan monooleate, n-alkylphenol ether ethoxylates, n-alkyl ether ethoxylates, and mixtures thereof.

14. A method of treating a human subject comprising a therapeutically effective amount of the pharmaceutical composition of claim 1 to the human subject in need of treatment thereof.

15. A transdermal delivery system comprising (i) an adhesive layer including the pharmaceutical composition of claim 1 blended within an adhesive material, and (ii) a backing layer.

16. A monolithic suspension-blend transdermal patch comprising: (a) a backing layer having inner and outer surfaces and being substantially impervious to the pharmaceutical composition of claim 1; (b) a first adhesive layer having a first surface covering at least a portion of the inner surface of the backing layer, the adhesive layer comprising a uniform suspension of a therapeutically-effective amount of the pharmaceutical composition in an adhesive; (c) a removable release liner in contact with a second surface of the adhesive layer.

17. A mixture comprising (i) a deep eutectic solvent, and (ii) a fungal biomass which has been cut, sheared, minced, or sliced, wherein the deep eutectic solvent is selected from the group consisting of a mixture of Choline Chloride and Glucose, a mixture of Choline Chloride and Fructose; a mixture of Choline chloride and ethylene glycol; a mixture of Choline chloride and 1,2-propanediol; a mixture of Choline Chloride and Glycerol; a mixture of Choline chloride and 1,4-butanediol; a mixture of Choline chloride and citric acid; a mixture of Choline Chloride and Malic Acid; a mixture of Choline chloride and Geranic acid; a mixture of Choline Chloride and Malonic Acid; a mixture of Choline Chloride and Urea; a mixture of Betaine and Glycerol; a mixture of Betaine and Citric acid; a mixture of Betaine and Malic acid; a mixture of Proline and Glycerol; a mixture of Lactic acid and glucose; and a mixture of and Choline chloride and Geranic Acid.

18. A liquid extracted from or filtered from the mixture of claim 17, wherein the liquid includes the deep eutectic solvent and one or more APIs derived from the fungal biomass, wherein the one or more APIs are selected from the group consisting of include baeocystin, norbaeocystin, N,N-dimethyltryptamine, 5-hydroxytryptamine (serotonin), 5-hydroxytryptophan, psilocybin and psilocin.

19. A method of treating a patient suffering from anxiety, depression, and/or other mental health conditions comprising contacting the skin of the patient in need of treatment thereof with the liquid of claim 18.

20. An adhesive blend comprising the liquid of claim 18 and an adhesive.
